(19) United States
(12) Patent Application Publication
(10) Pub. No.: US 2017/0164860 A1
(43) Pub. Date: Jun. 15, 2017

(54) LONG TERM PHYSIOLOGICAL SIGNAL SENSING PATCH

(71) Applicant: Sigknow Biomedical Corporation Limited, New Taipei City (TW)

(72) Inventors: Ming-wei Hung, New Taipei City (TW); Chih-Liang Wu, New Taipei City (TW); Yi-Yuan Chiu, New Taipei City (TW)

(73) Assignee: Sigknow Biomedical Corporation Limited

(21) Appl. No.: 15/094,665

(22) Filed: Apr. 8, 2016

(30) Foreign Application Priority Data
Dec. 14, 2015 (TW) 104141892

(51) Int. Cl. A61B 5/0424; A61B 5/0408; A61B 5/0492; A61B 5/00

(52) U.S. Cl. CPC A61B 5/0424; A61B 5/6833; A61B 5/04085; A61B 5/04087; A61B 5/0492; A61B 2562/182; A61B 2562/14; A61B 2560/0214

(57) ABSTRACT

A long term physiological signal sensing patch, including a water protective film, an electrode layer, a conductive hydrogel layer, a block layer and a first attaching layer. The water protective film provides functions of electrical insulation, stickiness and water protection. The electrode layer includes at least two electrodes attached to the bottom of the water protective film. The conductive hydrogel layer configured beneath the electrodes is electrically conductive and sticky. The block layer is under the electrodes to block water, and accommodates the conductive hydrogel layer. The first attaching layer is provided around the block layer. Therefore, the sensing patch can be attached to the human body for a long period of time to sense physiological signal, which is further transmitted to the external apparatus for more detailed analysis, calculation or display.

### CROSS-REFERENCE TO RELATED APPLICATION

This application claims the priority of Taiwanese patent application No. 104141892, filed on Dec. 14, 2015, which is incorporated herewith by reference.

### BACKGROUND OF THE INVENTION

1. Field of the Invention

The present invention generally relates to a long term physiological signal sensing patch, and more specifically to a long term physiological signal sensing patch provided with a water protection film for water protection to avoid falling off during shower while an user wears the long term physiological signal sensing patch, and a base used to support a circuit board and a top case so as to construct a wearable sensing device, thereby overcoming the problem in the prior arts that signal transmission wires have to be integrated and connected to connection device, and further improving convenience for use and carrying.

2. The Prior Arts

It is well known that various physical actions of the body generate specific electrical signals on the surface of the body. The electrical signals as physiological signals like electrocardiography (ECG), electroencephalogram (EEG), electromyography (EMG), ENG (electroneurogram), ERG (electroretinogram), EGG (electrogastrography), ENMG (electroneuromyography), ECoG (Electrocorticogram), EOG (Electrooculography), ENG (electronysagmography), and so forth can be measured or sensed by the measuring patch placed at the corresponding location of the body such as head, chest, abdomen, back and limb.

In the prior arts, a measuring patch is generally implemented by conductive metal pieces and tags provided on the attaching layer of the plastic foam. Further, the electrode sheets are attached to the bottom of the plastic foam, and the conductive hydrogel is attached to the bottom of the electrode sheets. The conductive metal pieces are electrically connected to the electrode sheets. In use, the conductive hydrogel directly contacts the human body, and signal lines are connected to the external device through the conductive metal pieces. Thus, the electrical signal on the human body can be measured by the electrode sheets via the conductive paste, and further transmitted to the external device through the electrode sheets, thereby performing specific analysis and display for physiological waveform, data and state.

While the measuring patch has a shape of thin sheet and is configured to directly attach to the human body and easily removed by peeling off for convenience in use, one of the drawbacks in the prior arts is that each pitch is intended for measuring only one physiological signal for one location on the human body, and in actual applications for multiple measurements, more patches and connection wires to connect the patches are needed and massively cover the human body. Thus, it is possible to carelessly peel off the patches or pull apart the connection wires. As a result, the doctor or the patient is quite puzzled and limited in regular action. In addition, the current patch lacks of water protection and is easily falling off due to sweat during exercise, and particularly is not suitable for bathing. It is inconvenient that the user should take off the patch before doing exercise or taking a bath. In general, the traditional patch is only applicable to static use and can not be worn for a long period of time. In other words, the patch in the prior art is not only very limited by application environment but also provides a short term for use.

Therefore, it is greatly needed to provide a new long term physiological signal sensing patch employing a water protection film to implement the function of water protection to prevent the patch from falling off while the user is taking a bath, and further utilizing a base to support a circuit board and a top case to form a wearable measuring or sensing device with portability and easy use, thereby overcoming the above problems in the prior arts that a number of signal wires are needed to integrate to connect with the external connection device.

### SUMMARY OF THE INVENTION

The primary objective of the present invention is to provide a long term physiological signal sensing patch, generally comprising a water protective film, an electrode layer, a conductive hydrogel layer, a block layer and a first attaching layer. The water protective film provides functions of electrical insulation, stickiness and water protection, and the electrode layer comprises at least two electrodes. The long term physiological signal sensing patch of the present invention is suitably attached to a human body for a long period of time to detect and measure a physiological signal such that an external electrical measuring instrument electrically connected to the long term physiological signal sensing patch performs more detailed analysis, calculation or display for the sensed physiological signal.

Further, the electrode layer is attached to the bottom of the water protection film, and the conductive hydrogel layer is configured beneath the corresponding electrodes, particularly at the end of the electrodes, so as to form electrical connection. The block layer is under the electrode layer, and has a hole for accommodating the conductive hydrogel layer.

The first attaching layer is around the block layer, and specifically beneath the electrode layer, and has a hole for accommodating the conductive hydrogel layer and the block layer.

The water protection film provides the functions of electrical insulation, stickiness and water protection, and is formed of a hydrophobic plastic material with adhesives coating. The area of the water protection film is configured larger than that of the electrode layer such that the electrode layer is fully covered with the water protection film, and the outer part of water protection film directly contacts the human body in use.

The electrode layer is formed of a conductive material to possess the function of signal transmission such that the electrodes are connected to the external electrical device like the circuit board or measuring instrument. For example, the conductive material generally comprises at least one of metal, conductive cloth, silver, silver chloride, graphite, conductive carbon, conductive silicone and conductive rubber. The conductive hydrogel layer is electrically conductive and sticky, and formed of resin, polyol, water and electrolyte or metal filler such that the upper surface of the conductive hydrogel layer is strongly adhered and fixed to the electrodes, and the lower surface of the conductive hydrogel layer is directly adhered to the human body, thereby transmitting the electrical signal of the human body to the electrodes.

The block layer effectively blocks vapor, and is formed of a material not or hardly absorbing water such as silicone, rubber or plastic foam. The first attaching layer is formed of a polymer adhesive material and provides high viscosity and weak absorbance so as to adhere to the human body. The first attaching layer also provides an effect of absorbing little liquid so as to avoid losing viscosity and prolong the attaching time.

More specifically, the conductive hydrogel layer and the block layer is provided in one of the configurations: the block layer is a ring, and surrounds contacts the conductive hydrogel layer; the block layer is a ring, and surrounds and partly contacts the conductive hydrogel layer; the block layer is a ring, and surrounds but does not contact the conductive hydrogel layer; the block layer is at least two segmental pieces surrounding and contacting the conductive hydrogel layer; the block layer is at least two segmental pieces surrounding and partly contacting the conductive hydrogel layer; and the block layer is at least two segmental pieces surrounding but not contacting the conductive hydrogel layer.

Moreover, the water protection film further comprises at least two holes, which are configured to align with and correspond to the electrodes such that the electrodes are exposed. Or alternatively, the electrodes directly penetrate through the corresponding holes of the water protection film. As a result, the sensed physiological signal is transmitted to an external connection device via the electrode layer.

The present invention further comprises a top case, a water-proof ring, a support layer, a reinforcing layer, a base, a second attaching layer and a release layer, and particularly, the top case, the water-proof ring and the support layer are sequential from top to bottom stacked on the water protection film. The support layer provides sufficient mechanical strength to increase stiffness of the water protection film. The water-proof ring is sandwiched between the top case and the water protection film for providing a water-tight function to isolate and prevent water from seeping into. The reinforcing layer is under the electrode layer and has high tensile strength and toughness. The reinforcing layer has at least two holes for accommodating the electrodes. The base is configured under the reinforcing layer and combined with the top case to form a closed and water-tight accommodation room for supporting the circuit board and/or battery. The built-in battery supplies electric power to meet the requirement of long term operation for measuring and recording. In addition, the second attaching layer is configured under the base for enhancing persistent adhesion, and the release layer is attached to the bottom of the water protection film and the conductive hydrogel layer such that a closed room is formed by the release layer and the water protection film to prevent internal absorbed water in a form of gel from evaporating. Since the release layer is at the bottom of the patch of the present invention, any pollutant is kept off to maintain sufficient stickiness as desired. In use, the release layer is first removed or peeled off so as to expose the water protection film, the conductive hydrogel layer, the first attaching layer and the second attaching layer for directly being attached to the human body. Particularly, the base is configured to support the circuit board and the top case, and the circuit board is electrically connected to the electrodes, thereby forming a wearable measuring device for easy use.

Therefore, only one patch of the present invention is needed to simultaneously measure or sense ECG and EMG as the physiological signal of the human body. In particular, when the user is taking exercise and sweating a little, the first attaching layer configured to absorb little sweat helps prolong the use time without limitation to the application environment.

### BRIEF DESCRIPTION OF THE DRAWINGS

The present invention can be understood in more detail by reading the subsequent detailed description in conjunction with the examples and references made to the accompanying drawings, wherein:

FIG. 1 is a cross sectional view showing a long term physiological signal sensing patch according to one embodiment of the present invention;

FIG. 2 is an exploded view showing the long term physiological signal sensing patch of the present invention; and

FIG. 3 is a top view showing the long term physiological signal sensing patch according to one embodiment of the present invention.

### DETAILED DESCRIPTION OF THE PREFERRED EMBODIMENT

The present invention may be embodied in various forms and the details of the preferred embodiments of the present invention will be described in the subsequent content with reference to the accompanying drawings. The drawings (not to scale) show and depict only the preferred embodiments of the invention and shall not be considered as limitations to the scope of the present invention. Modifications of the shape of the present invention shall too be considered to be within the spirit of the present invention.

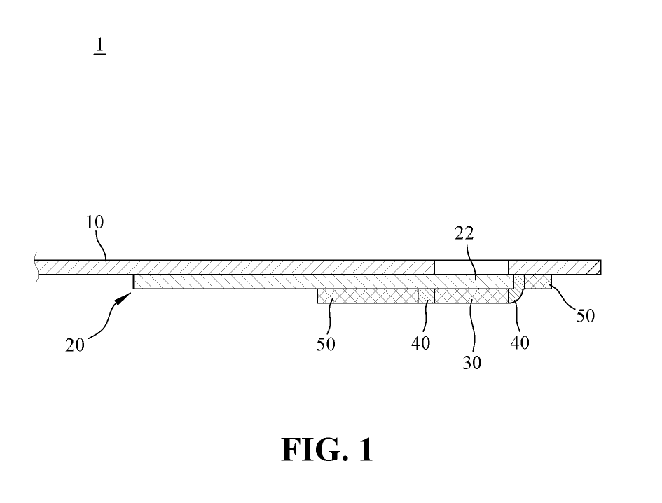

Please refer to FIG. 1 illustrating the long term physiological signal sensing patch according to one embodiment of the present invention. As shown in FIG. 1, the long term physiological signal sensing patch 1 of the present invention generally comprises a water protective film 10, an electrode layer 20, a conductive hydrogel layer 30, a block layer 40 and a first attaching layer 50. The water protective film 10 provides functions of electrical insulation, stickiness and water protection. The electrode layer 20 comprises at least two electrodes 22, which can be configured to be independent and not connected together. The long term physiological signal sensing patch 1 of the present invention is suitably attached to a human body for a long period of time to detect and measure a physiological signal, and an external electrical instrument electrically connected to the long term physiological signal sensing patch 1 thus performs more detailed analysis, calculation or display for the sensed physiological signal.

Specifically, the electrodes 22 of the electrode layer 20 are attached to the bottom of the water protection film 10 to provide a function of electrical signal transmission. The conductive hydrogel layer 30 is electrically conductive and sticky, and configured beneath the corresponding electrodes 22, particularly at the end of the electrodes 22, so as to form electrical connection. The block layer 40 is under the electrode layer 20, and has a hole for accommodating the conductive hydrogel layer 30. Moreover, the block layer 40 is attached to the bottom of the electrodes 22 such that one electrode corresponds to one block layer.

Further, the first attaching layer 50 is around the block layer 40, and specifically beneath the electrode layer 20, and has a hole for accommodating the conductive hydrogel layer 30 and the block layer 40.

More specifically, the water protection film 10 provides the functions of electrical insulation, stickiness and water protection, and is formed of a hydrophobic plastic material with adhesives coating. The area of the water protection film 10 is configured larger than that of the electrode layer 20 such that the electrode layer 20 is fully covered with the water protection film 10, and the outer part of water protection film 10 directly contacts the human body in use.

The electrode layer 20 is formed of a conductive material, such as metal, conductive cloth, silver, silver chloride, graphite, conductive carbon, conductive silicone or conductive rubber. The electrode 22 of the electrode layer 20 is partly or fully connected to the external electrical device like circuit board or measuring device. For example, the measuring device is a processing unit, microcontroller, computer or server. The conductive hydrogel layer 30 is electrically conductive and sticky, and formed of resin, polyol, water and electrolyte or metal filler such that the upper surface of the conductive hydrogel layer 30 is strongly adhered and fixed to the electrode 22, and the lower surface of the conductive hydrogel layer 30 is directly adhered to the human body, thereby transmitting the electrical signal of the human body is to the electrode 22.

The block layer 40 effectively blocks vapor, and is formed of a material not or hardly absorbing water such as silicone, rubber or plastic foam. The first attaching layer 50 is formed of a polymer adhesive material and provides high viscosity and weak absorbance. For instance, the polymer adhesive material may comprise scar-removing paste, hydrocolloid adhesive, polyurethane (PU), silicone or hydrogel, which well adheres to the human body. Furthermore, the first attaching layer 50 also provides an effect of absorbing little liquid so as to avoid losing viscosity and prolong the attaching time.

Therefore, when the long term physiological signal sensing patch 1 of the present invention is in use, the conductive hydrogel layer 30 is directly attached to the human body like chest or head, and the electrodes 22 of the electrode layer 20 sense the electrical signal of the human body serving as the physiological signal, thereby achieving a patch function for measuring the physiological signal. In particular, the first attaching layer 50 provides primary adhesion to the human body, and the outer part of the sticky water protection film 10 is also attached to the human body for additional adhesion so as to greatly improve overall stability.

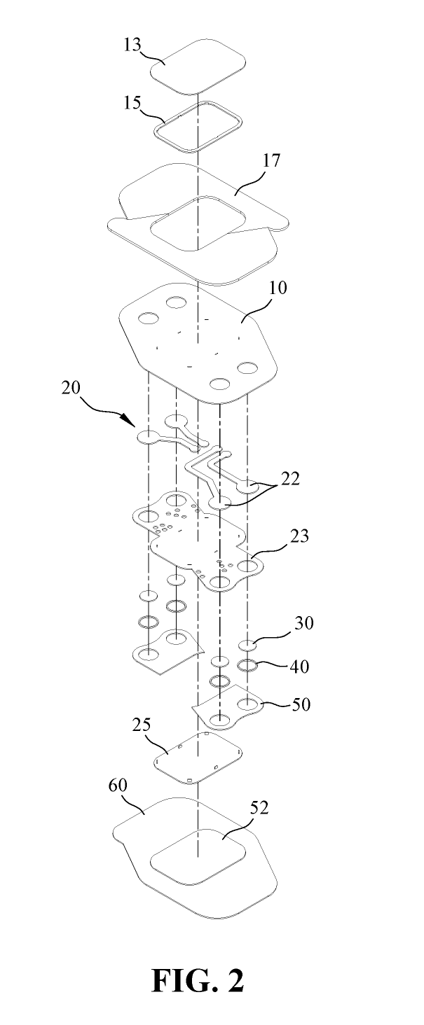

To enhance the patch function and improve convenience for use, further refer to FIG. 2 showing the exploded view of the long term physiological signal sensing patch of the present invention. As shown in FIG. 2, the long term physiological signal sensing patch 1 of the present invention may further comprises a top case 13, a water-proof ring 15 and a support layer 17, which are sequential from top to bottom stacked on the water protection film 10. The water-proof ring 15 is sandwiched between the top case 13 and the water protection film 10 for providing a water-tight function to isolate and prevent water from seeping into. The support layer 17 provides sufficient mechanical strength to increase stiffness of the water protection film 10. As a result, it is easy for the user to attach the long term physiological signal sensing patch 1 to the human body, and then the support layer 17 is removed. In addition, the support layer 17 and the water protection film 10 are combined through a non-glue means like hot pressing, to avoid any glue remaining on the water protection film 10 after the support layer 17 is removed.

The long term physiological signal sensing patch 1 of the present invention may further comprise a reinforcing layer 23 and a base 25. The reinforcing layer 23 is under the electrode layer 20 and has high tensile strength and toughness. The reinforcing layer 23 has at least two holes for accommodating the electrodes 22 to strengthen the electrode layer 20. The base 25 is configured under the reinforcing layer 23 and combined with the top case 13 to form a closed and water-tight accommodation room for supporting the circuit board and/or battery, and the circuit board is electrically connected to the electrodes so as to form a portable measuring device for physiological signal. The built-in battery supplies electric power to meet the requirement of long term operation for measuring and recording.

Additionally, the above reinforcing layer 23 has an area larger than the electrode layer 20, and the electrodes 22 are tightly attached around the least two holes of the reinforcing layer 23. Thus, sweat from the human body surely does not seep into the electrodes 22 to get rid of noise interference. The reinforcing layer 23 may further have more additional holes to improve overall vapor permeability. The base 25 is preferably provided with snaps or pillars, which penetrate the reinforcing layer 23 to help tightly joint the base 23 and the top case 13. Next, the first attaching layer 50 is implemented by two double-sided paste patches horizontally attached to two sides of the reinforcing layer 23, respectively, and each double-sided paste patch has holes for accommodating the block layer 40 and the conductive hydrogel layer 30.

Moreover, the present invention comprises the second attaching layer 52 or the release layer 60. The second attaching layer 52 is configured under the base 25 for enhancing persistent adhesion, and the release layer 60 is attached to the bottom of the water protection film 10 and the conductive hydrogel layer 30 such that a closed room is formed by the release layer 60 and the water protection film 10 to prevent internal absorbed water in a form of gel from evaporating. The release layer 60 is specifically configured at the bottom of the long term physiological signal sensing patch 1 to avoid pollution and maintain sufficient adhesion. Thus, the area of the release layer 60 is preferably larger than that of the water protection film 10. Since the water protection film 10, the conductive hydrogel layer 30, the first attaching layer 50 and the second attaching layer 52 possibly have different thickness and the corresponding stage differences are formed, the release layer 60 can meet the stage differences through appropriately shaping. In particular, water-tight effect is achieved by the release layer 60 and the water protection film 10. In use, the release layer 60 is first removed or peeled off so as to expose the water protection film 10, the conductive hydrogel layer 30, the first attaching layer 50 and the second attaching layer 52 for directly being attached to the human body.

It is preferred that the second attaching layer 52 is formed of the polymer adhesive material with high viscosity and weak absorbance like scar-removing paste, artificial skin, PU, silicone or hydrogel. The release layer 60 can be implemented by a substrate formed of paper, a separator with water and solvent endurance and a release agent coating layer, wherein the separator is provided on the surface of the substrate, and the release agent coating layer covers the separator.

Further, the conductive hydrogel layer 30 is configured at the same horizontal surface of the electrode layer 20 like the lower surface, and particularly, does not overlap or interfere with the reinforcing layer 23. The conductive hydrogel layer 30 may have a shape of circle, square or rhombus. The block layer 40 is under the electrode layer 20 and thus does not overlap or interfere with the reinforcing layer 23.

More specifically, the conductive hydrogel layer 30 and the block layer 40 can be provided in one of the configurations as following: (1) in the first configuration, the block layer 40 is a ring, and surrounds contacts the conductive hydrogel layer 30; (2) in the second configuration, the block layer 40 is a ring, and surrounds and partly contacts the conductive hydrogel layer 30; (3) in the third configuration, the block layer 40 is a ring, and surrounds but does not contact the conductive hydrogel layer 30; (4) in the fourth configuration, the block layer 40 is at least two segmental pieces surrounding and contacting the conductive hydrogel layer 30; (5) in the fifth configuration, the block layer 40 is at least two segmental pieces surrounding and partly contacting the conductive hydrogel layer 30; and (6) in the sixth configuration, the block layer 40 is at least two segmental pieces surrounding but not contacting the conductive hydrogel layer 30.

In addition, the water protection film 10 further comprises at least two holes, which are configured to align with and correspond to the at least two electrodes 22, particularly the ends of the electrodes 22, such that the electrodes 22 are exposed. Alternatively, the at least two electrodes 22 are configured to directly penetrate through the corresponding holes of the water protection film 10. As a result, the sensed physiological signal is transmitted to an external connection device via the electrode layer 20.

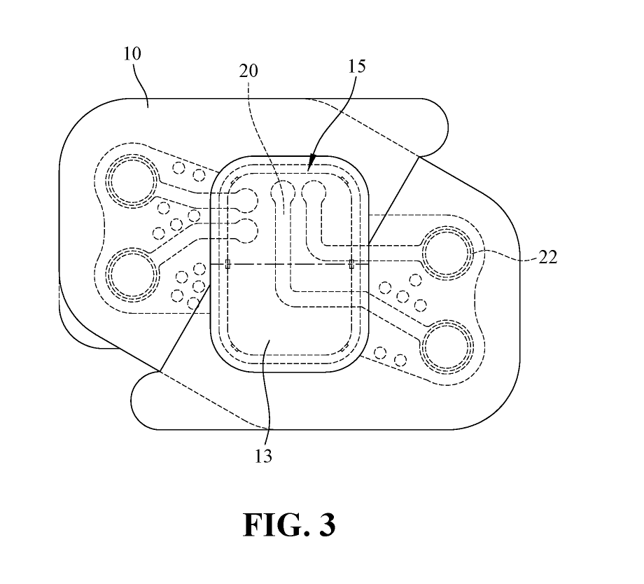

Further refer to FIG. 3 illustrating the long term physiological signal sensing patch according to one embodiment of the present invention. The long term physiological signal sensing patch 1 of the present invention has a shape of thin sheet and is fully attached to the human body. Particularly, the present invention may integrate a plurality of electrode layers in the same long term physiological signal sensing patch 1, which allows the user to achieve the object of sensing physiological signal for a long period of time. In contrast, the user needs at least two patches in the prior arts attached to the human body for the same object. Obviously, the present invention indeed greatly improves convenience for use.

Additionally, since the present invention has larger area due to integration of the electrode layers, the whole patch may have weaker mechanical strength and easily deform. The support layer is thus used to enhance overall stiffness of the patch so as to improve attaching operation.

For example, the user attaches the present invention to the left chest of the human body for ECG, or to the chest or abdomen for measuring respiration. Alternatively, the present invention can be provided at different position of the human body for corresponding physiological measurements, such as EEG, EMG, ENG ,ERG, EGG, ENMG, ECoG, EOG, ENG, and so forth. The present invention particularly provides excellent water protection such that the user can wears the patch during bathing, exercising or even sleeping without worrying about the patch falling off

The water protection film 10 possesses good adhesion and appropriate water protection, and has a thickness of about 0.01˜0.07 mm so as to keep sufficient vapor permeability and prevent the patch from falling off when the user sweats due to exercise or sultriness. The thickness of the electrode layer is within 0.01˜0.3 mm to maintain the whole patch soft, and the attaching time for the patch is effectively prolonged. Additionally, the holes of the surface of the water protection film 10 are intended to accommodate and partly expose the electrode layer 20 so as to connect the external measuring instrument. The effect of absorbing little liquid provided by the first attaching layer 50 can avoid losing adhesion due to sweat, and further prolong the attaching time for the human body. Overall, the attaching effect of the present invention generally lasts for at least 7 days.

Through the electrode layer 20, the electrical signal is transmitted. For instance, the signal of ECG is electrically transmitted to the external measuring instrument, which performs analysis, calculation or display. Moreover, the electrode layer 20 can be used to measure or sense respiration signal in impedance pneumography, which imposes a carrier signal with high frequency and low current into the human body to measure respiration signal like respiration waveform, respiration rate, and so on. Therefore, the electrode layer 20 can be further provided with four electrodes to achieve synchronization for measuring ECG and respiration signal, wherein two of the four electrodes are intended for measuring ECG and the other two electrodes for respiration signal.

The block layer 40 of the present invention is configured at the outer side of the conductive hydrogel layer 30 to implement the blocking effect and prevent the conductive hydrogel layer 30 from dehydration due to long term contact with the absorbing materials in the surroundings.

From the above mention, one aspect of the present invention is that the user needs only one patch of the present invention, but at least two patches are needed in the prior arts to achieve the desired function of measuring two kinds of physiological signal like ECG and EMG The stiffness of the patch is enhanced by the support layer.

Additionally, since the first attaching layer can absorb little liquid such as sweat, the attaching time of the patch is prolonged when the user is doing exercise. However, the traditional patch fails to work while the user wears the patch is bathing or exercising, and is only effective for use in static circumstance.

The block layer of the repent invention prevents the conductive hydrogel layer from influence by the surrounding material like the first attaching layer, which absorbs water in the conductive hydrogel layer and seriously affects the measuring function and the effective period of time for use. Or, the block layer stops pollution caused by the surrounding material, and avoids losing the desired properties. In particular, it is well known that no block layer is provided in the prior arts.

Furthermore, the present invention provide water protection to make sure the pact does not fall off during bathing, but the prior arts fail to achieve the same effect. Particularly, the base is used to support the circuit board and the top case, and the circuit board and the electrodes are electrically connected to form the wearable measuring device. However, the prior arts have to integrate lots of signal wires to connect with the connection device, and it is thus inconvenient for portable use.

Although the present invention has been described with reference to the preferred embodiments, it will be understood that the present invention is not limited to the details described thereof. Various substitutions and modifications have been suggested in the foregoing description, and others will occur to those of ordinary skill in the art. Therefore, all such substitutions and modifications are intended to be embraced within the scope of the invention as defined in the appended claims.

What is claimed is:

1. A long term physiological signal sensing patch for being directly attached to a human body to sense an electrical signal on a surface of the human body as a physiological signal, comprising:
a water protective film providing functions of electrical insulation, stickiness and water protection;
an electrode layer providing a function of electrical signal transmission, and having at least two electrodes attached to a bottom of the water protective film;
a conductive hydrogel layer electrically conductive and sticky, configured beneath the electrodes to provide electrical connection;
a block layer configured under the electrodes for blocking vapor and having a hole for accommodating the conductive hydrogel layer, and
a first attaching layer configured around the block layer,
wherein the first attaching layer is located beneath the electrodes and has at least two holes for accommodating the block layer and the conductive hydrogel layer.

2. The long term physiological signal sensing patch as claimed in claim 1, wherein the water protective film is formed of a hydrophobic plastic material with adhesives coating, the electrode layer is formed of a conductive material, the conductive material comprises metal, conductive cloth, silver, silver chloride, graphite, conductive carbon, conductive silicone or conductive rubber, the conductive hydrogel layer is formed of resin, electrolyte or metal filler, and the block layer is formed of a material not or hardly absorbing water.

3. The long term physiological signal sensing patch as claimed in claim 1, wherein the first attaching layer is formed of a polymer adhesive material and provides high viscosity and weak absorbance, and the polymer adhesive material comprises scar-removing paste, artificial skin, polyurethane (PU), silicone or hydrogel.

4. The long term physiological signal sensing patch as claimed in claim 1, wherein the conductive hydrogel layer and the block layer are provided in one of six configurations to prevent the conductive hydrogel layer from losing electrical conduction due to contacting the first attaching layer for a long period of time, and the six configurations comprise:
a first configuration, in which the block layer is a ring, and surrounds contacts the conductive hydrogel layer;
a second configuration, in which the block layer is a ring, and surrounds and partly contacts the conductive hydrogel layer;
a third configuration, in which the block layer is a ring, and surrounds but does not contact the conductive hydrogel layer;
a fourth configuration, in which the block layer is at least two segmental pieces surrounding and contacting the conductive hydrogel layer;
a fifth configuration, in which the block layer is at least two segmental pieces surrounding and partly contacting the conductive hydrogel layer; and
a sixth configuration, in which the block layer is at least two segmental pieces surrounding but not contacting the conductive hydrogel layer.

5. The long term physiological signal sensing patch as claimed in claim 1, further comprising a reinforcing layer with high tensile strength and toughness, wherein the reinforcing layer is under the electrodes and has at least two holes for accommodating the electrodes.

6. The long term physiological signal sensing patch as claimed in claim 1, further comprising a support layer and a release layer, wherein the support layer provides mechanical strength and is attached to the water protection film for increasing strength of the water protection film, and intended to remove after the long term physiological signal sensing patch is attached to the human body, the release layer is attached to and under the water protection film and the conductive hydrogel layer, the release layer and the water protection film are tightly sealed to form a closed room, and the water protection film, the conductive hydrogel layer and the first attaching layer are exposed and directly attached to the human body after the release layer is removed.

7. The long term physiological signal sensing patch as claimed in claim 5, further comprising a top case, a base, a water-proof ring and a second attaching layer, wherein the top case and the water-proof ring are sequentially from top to bottom stacked on the water protection film, the water-proof ring is sandwiched between the top case and the water protection film for providing a water-tight function, the base is configured under the reinforcing layer and combined with the top case to form a closed and water-tight accommodating room for support a circuit board and/or a battery, the battery supplies power, and the second attaching layer is attached to a bottom of the base to enhance lasting adhesion.

8. The long term physiological signal sensing patch as claimed in claim 6, wherein the release layer has an area larger than an area of the water protection film.

9. The long term physiological signal sensing patch as claimed in claim 7, wherein the second attaching layer is formed of a polymer adhesive material and provides high viscosity and weak absorbance, and the polymer adhesive material comprises scar-removing paste, artificial skin, polyurethane (PU), silicone or hydrogel.

10. The long term physiological signal sensing patch as claimed in claim 1, wherein the water protection film further comprises at least two holes, the at least two electrodes are configured to align with and correspond to the at least two holes so as to expose, or alternatively, the at least two electrodes are configured to directly penetrate through the corresponding holes of the water protection film so as to transmit the sensed physiological signal to an external connection device via the electrode layer, and the external connection device comprises a circuit board or a measuring instrument.
